(19) United States
(12) Patent Application Publication
(10) Pub. No.: US 2025/0210179 A1
(43) Pub. Date: Jun. 26, 2025

(54) STATE ESTIMATION APPARATUS, STATE ESTIMATION METHOD, AND NON-TRANSITORY COMPUTER-READABLE MEDIUM

(71) Applicant: Yokogawa Electric Corporation, Tokyo (JP)

(72) Inventors: Kiyomi MURAOKA, Tokyo (JP); Motohiko ISHIDA, Tokyo (JP); Hirotaka YAMASHITA, Tokyo (JP); Takashi TSUBOTA, Tokyo (JP)

(21) Appl. No.: 18/928,141

(22) Filed: Oct. 27, 2024

(30) Foreign Application Priority Data
Dec. 26, 2023 (JP) 2023-219220

(51) Int. Cl. G16H 20/70

(52) U.S. Cl. CPC G16H 20/70

(57) ABSTRACT

Provided is a state estimation apparatus including an information acquisition unit which acquires brain wave information of a target person to whom a content is provided, and a state estimation unit which estimates a state of the target person to whom the content is provided, based on the brain wave information of the target person. The information acquisition unit may further acquire biometric information of the target person to whom the content is provided. The state estimation unit may estimate the state based on the brain wave information and the biometric information. The information acquisition unit may acquire the brain wave information of the target person before and while the content is provided. The state estimation unit may estimate the state based on a change from the brain wave information before the content is provided, to the brain wave information while the content is provided, and on the biometric information.

The contents of the following patent application(s) are incorporated herein by reference: NO. 2023-219220 filed in JP on Dec. 26, 2023

### BACKGROUND

### 1. Technical Field

The present invention relates to a state estimation apparatus, a state estimation method, and a non-transitory computer-readable medium.

### 2. Related Art

Patent document 1 describes that “in the learning phase, . . . first brain activity data indicating an amount of variation in an amount of brain activity is acquired” (in claim 1).

### PRIOR ART DOCUMENT

### Patent Document

Patent Document 1: Japanese Patent No. 6935774

### BRIEF DESCRIPTION OF THE DRAWINGS

FIG. 1 is a diagram showing an example of a situation in which a content 120 is provided to a target person 110.

FIG. 2 is a diagram showing another example of the situation in which the content 120 is provided to the target person 110.

FIG. 3 is a diagram showing another example of the situation in which the content 120 is provided to the target person 110.

FIG. 4 is a block diagram showing an example of a state estimation apparatus 100 according to an embodiment of the present invention.

FIG. 5 is a diagram showing an example of an information acquisition unit 10.

FIG. 6 is a diagram showing an example of a state S of the target person 110, estimated by a state estimation unit 20.

FIG. 7 is a diagram showing another example of the situation in which the content 120 is provided to the target person 110.

FIG. 8 is a diagram showing an example of a relationship between the state S, a test result Rt and a state of understanding Cs.

FIG. 9 is a diagram showing an example of a state of understanding inference model 72.

FIG. 10 is a flowchart showing an example of a state estimation method according to an embodiment of the present invention.

FIG. 11 is a diagram showing an example of a computer 2200, in which the state estimation apparatus 100 according to an embodiment of the present invention may be embodied in whole or in part.

### DESCRIPTION OF EXEMPLARY EMBODIMENTS

Hereinafter, the present invention will be described through embodiments of the invention, but the following embodiments do not limit the invention according to the claims. In addition, not all of the combinations of features described in the embodiments are essential to the solution of the invention.

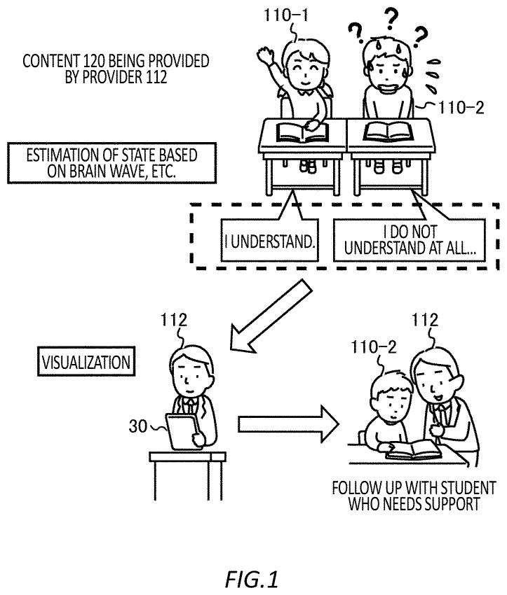

FIG. 1 is a diagram showing an example of a situation in which a content 120 is provided to a target person 110. The content 120 is information to be experienced or information to be viewed by the target person 110. “Experience” herein refers to participating in a seminar, etc. at a venue of the seminar, etc., and “view” refers to receiving video and audio of the seminar, etc. provided via a terminal 130 (described below). References herein to “view” may include both “view” and “experience,” and references herein to “experience” may include both “view” and “experience. The information to be viewed or experienced includes at least one of visual information or audio information.

FIG. 1 is an example of a case where a provider 112 provides the content 120 to the target person 110 in a school, a cram school, or the like. In the example in FIG. 1, the target person 110 is a child, the provider 112 is a teacher at the school, the cram school, or the like, and the content 120 is a lesson at the school, the cram school, or the like. In the example in FIG. 1, the provider 112 provides the content 120 to two target persons 110 (a target person 110-1 and a target person 110-2) in a classroom. In the example in FIG. 1, the target person 110-1 feels that he/she “understands” the content 120, while the target person 110-2 feels that he/she “does not understand the content 120 at all.”

A providing unit 30 provides a state S of the target person 110. As described below, the state S is estimated based on brain wave information Ib (described below) of the target person 110. The providing unit 30 is, for example, a tablet, a display, a monitor, or the like. The provider 112 may be a user of a state estimation apparatus 100. In the example in FIG. 1, the provider 112 confirms the state S of the target person 110 using the providing unit 30.

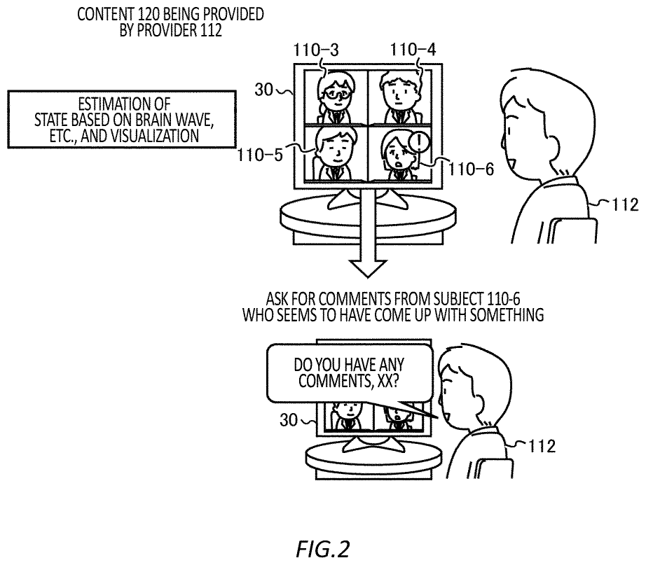

FIG. 2 is a diagram showing another example of the situation in which the content 120 is provided to the target person 110. FIG. 2 is an example of a case where the provider 112 provides the content 120 to the target person 110 in an online meeting at a workplace or the like. In the example in FIG. 2, the target person 110 is a participant in the meeting, the provider 112 is a host of the meeting, and the content 120 is a content explained in the meeting.

In the example in FIG. 2, the provider 112 provides the content 120 to four target persons 110 (a target person 110-3 to a target person 110-6). The example in FIG. 2 shows a state in which the target person 110-6 has come up with something. In the example in FIG. 2, the provider 112 confirms that the target person 110-6 is in a state in which he/she has come up with something, based on the state S provided by the providing unit 30. In the example in FIG. 2, the provider 112 is asking the target person 110-6, “Do you have any comments?”

In the example in FIG. 2, the target person 110 is displayed on the providing unit 30 and the state S is provided. The state S may be provided by the providing unit 30 such as the tablet. When the state S is provided by the providing unit 30 such as the tablet, the target person 110 may be displayed on the display, the monitor, or the like, separate from the providing unit 30.

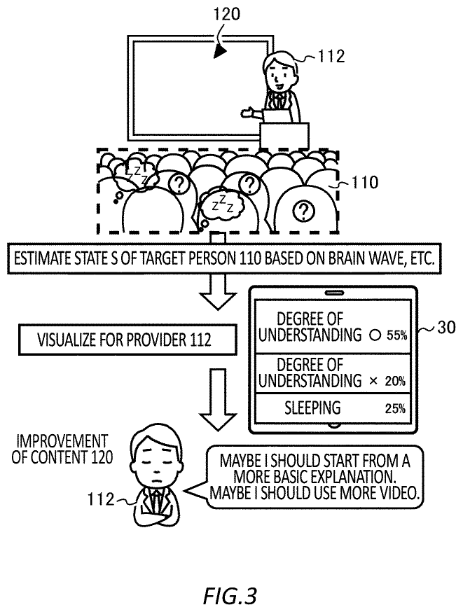

FIG. 3 is a diagram showing another example of the situation in which the content 120 is provided to the target person 110. FIG. 3 is an example of a case where the provider 112 provides the content 120 to a plurality of target persons 110 in the seminar. In the example in FIG. 3, the target person 110 is a participant in the seminar, the provider 112 is a lecturer of the seminar, and the content 120 is a content explained in the seminar.

A state of the target person 110 which reflects a psychological state of the target person 110 is referred to as the state S. The state S includes a potential state of the target person 110. The potential state of the target person 110 refers to a state which reflects the target person 110's own psychological state, of which the target person 110 himself/herself is not aware. For example, if the target person 110 feels that he/she cannot understand the content 120, the state S may reflect the psychological state of the target person 110 that he/she cannot understand the content. In the example in FIG. 3, the providing unit 30 is provided with a degree of understanding of the plurality of target persons 110. In the example in FIG. 3, the degree of understanding is classified into stages, and the percentage of the target persons 110 who fall into each of the stages of the degree of understanding is provided. In the example in FIG. 3, based on the degree of understanding provided by the providing unit 30, the provider 112 thinks, “Maybe I should start from a more basic explanation. Maybe I should use more video.”

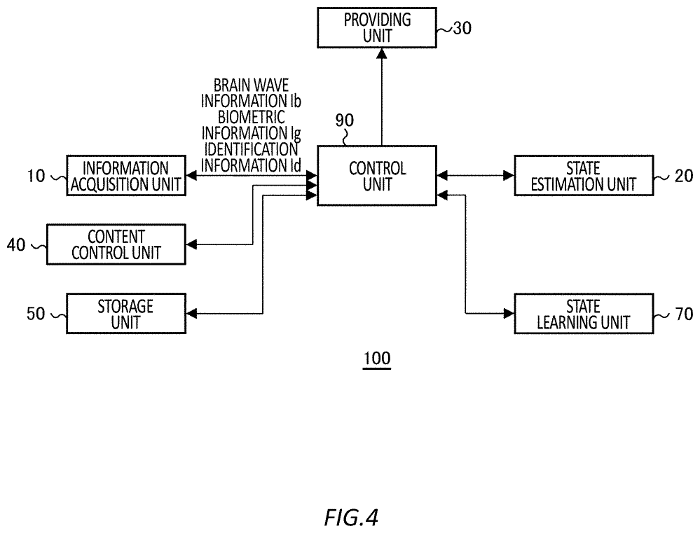

FIG. 4 is a block diagram showing an example of the state estimation apparatus 100 according to an embodiment of the present invention. The state estimation apparatus 100 includes an information acquisition unit 10 and a state estimation unit 20. The state estimation apparatus 100 may include the providing unit 30, a content control unit 40, a storage unit 50, a state learning unit 70, and a control unit 90.

A part or a whole of the state estimation apparatus 100 may be realized by a computer. The control unit 90 may be a Central Processing Unit (CPU) of the computer. When the state estimation apparatus 100 is realized by the computer, an evaluation assistance program for causing the computer to function as the state estimation apparatus 100 may be installed on the computer, or an information processing program for causing the computer to execute an information processing method described below may be installed on the computer.

The information acquisition unit 10 acquires the brain wave information of the target person 110 to whom the content 120 is provided. The brain wave information of the target person 110 is referred to as the brain wave information Ib. The brain wave information Ib of the target person 110 to whom the content 120 is provided refers to the brain wave information Ib of the target person 110 while the content 120 is provided. The information acquisition unit 10 may further acquire the brain wave information Ib of the target person 110 before the content 120 is provided.

The brain wave information Ib may be information which reproduces at least a part of a temporal waveform of a brain wave of the target person 110. The brain wave information Ib may include data obtained by sampling the temporal waveform of the brain wave, may include data indicating a magnitude of frequency components of the brain wave at one or more frequencies, or may include another piece of data. For example, the brain wave information Ib includes data indicating a magnitude of at least one component of a delta wave (less than 4 Hz), a theta wave (4 Hz or more and less than 8 Hz), an alpha wave (8 Hz or more and less than 14 Hz), a beta wave (14 Hz or more and less than 26 Hz), or a gamma wave (26 Hz or more and less than 40 Hz).

The alpha wave may be further classified into a low alpha wave (8 Hz or more and less than 10 Hz), a medium alpha wave (10 Hz or more and less than 12 Hz), and a high alpha wave (12 Hz or more and less than 14 Hz) according to a frequency band. The brain wave information Ib may include data indicating a magnitude of at least one of the low alpha wave, the medium alpha wave, or the high alpha wave.

The beta wave may be further classified into a low beta wave (14 Hz or more and less than 18 Hz) and a high beta wave (18 Hz or more and less than 26 Hz) according to the frequency band. The brain wave information Ib may include data indicating a magnitude of at least one of the low beta wave or the high beta wave.

The brain wave information Ib may include information on the temporal waveform of one or more brain waves measured at one or more positions on a head part of including a head and a face of the target person 110. For example, the brain wave information Ib may be acquired by measuring the temporal waveform of potentials of electrodes arranged at equal intervals in a vicinity of a scalp of the target person 110, as in the international 10-20 system, or may be acquired by another method. The intervals of a plurality of electrodes which are arranged on the scalp may not be equal. The electrodes may be provided on a wearable appliance which is worn on the head part of the target person 110, such as a headgear, a headphone, earphones, glasses, or the like. The brain wave information Ib may be information obtained by acquiring electric signals at the electrodes embedded in a body of the target person 110, through wireless communication.

A sum of an amplitude of the alpha wave, an amplitude of the beta wave, an amplitude of the theta wave, an amplitude of the gamma wave, and an amplitude of the delta wave at certain timing is referred to as a total amplitude As. As an example, in a case where a proportion of the amplitude of the delta wave of the target person 110 to the total amplitude As is greater than any of a proportion of the amplitude of the alpha wave to the total amplitude As, a proportion of the amplitude of the beta wave to the total amplitude As, a proportion of the amplitude of the theta wave to the total amplitude As, and a proportion of the amplitude of the gamma wave to the total amplitude As, it can be inferred that the target person 110 is in a sleeping state.

The state estimation unit 20 estimates the state S of the target person 110 to whom the content 120 is provided, based on the brain wave information Ib of the target person 110. The providing unit 30 may provide the state S to the provider 112. This allows the provider 112 to recognize the state S of the target person 110. The provider 112 can control a manner in which the content 120 progresses, by recognizing the state S of the target person 110. For example, when the target person 110 is in the state S in which the degree of understanding is low or is decreasing, the provider 112 may stop progression of the content 120, and can provide additional explanation on the content 120 to increase the degree of understanding. For example, when the target person 110 is in the state S in which the degree of understanding is high or is increasing, the provider 112 can continue progressing through the content while checking the degree of understanding.

The brain wave information Ib may reflect the state S based on the degree of understanding of the content 120 by the target person 110. The state estimation apparatus 100 estimates the state S based on the brain wave information Ib of the target person 110. Therefore, the user of the state estimation apparatus 100 (e.g., the provider 112) can recognize the state S of the target person 110.

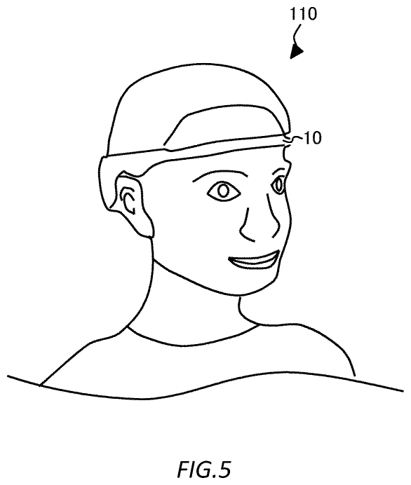

FIG. 5 is a diagram showing an example of an information acquisition unit 10. The information acquisition unit 10 may have an electroencephalograph which can measure the brain wave information Ib, or may have a communication device which acquires the brain wave information Ib measured by an external electroencephalograph. The information acquisition unit 10 of the present example is an electroencephalograph of a headgear type. The information acquisition unit 10 may be an electroencephalograph of an earphone type. In the present example, the target person 110 may be provided with the content 120, in a state in which the electroencephalograph of the headgear type or the earphone type is worn. In this manner, the information acquisition unit 10 acquires the brain wave information Ib of the target person 110 in a state in which the content 120 is provided.

If the information acquisition unit 10 is the electroencephalograph of the headgear type, the content control unit 40 and the control unit 90 may not be housed in the housing of the headgear. The brain wave information Ib acquired by the information acquisition unit 10 may be transmitted wirelessly to the control unit 90.

The information acquisition unit 10 may further acquire biometric information of the target person 110 to whom the content 120 is provided. The biometric information is referred to as biometric information Ig.

The information acquisition unit 10 may further acquire the biometric information Ig before the content 120 is provided. The biometric information Ig may include at least one of information on a heart rate, information on an amount of perspiration, or information on body temperature of the target person 110. The biometric information Ig of the target person 110 may be acquired by a sensor provided in the wearable appliance worn by the target person 110.

The state estimation unit 20 may estimate the state S based on the brain wave information Ib and the biometric information Ig. The state estimation unit 20 may estimate the state S of the target person 110 based on a change from the brain wave information Ib before the content 120 is provided to the brain wave information Ib while the content 120 is provided, and based on the biometric information Ig.

In the heart rate of the target person 110, a magnitude of a first power spectrum is referred to as LF, and a magnitude of a second power spectrum is referred to as HF. A frequency band of the second power spectrum is a band in which a frequency is higher than that in a frequency band of the first power spectrum. The frequency band of the first power spectrum and the frequency band of the second power spectrum may not overlap each other. The frequency band of the first power spectrum is, for example, 0.04 Hz to 0.15 Hz. The frequency band of the second power spectrum is, for example, 0.15 Hz to 0.4 Hz.

A change from a proportion of the amplitude of the brain wave in a predetermined frequency band to the total amplitude As in the brain wave information Ib before the content 120 is provided, to the proportion of the amplitude of the brain wave in the predetermined frequency band to the total amplitude As in the brain wave information Ib while the content 120 is provided, is referred to as a change C. The state estimation unit 20 may estimate the state S based on the change C and the ratio of LF to HF (LF/HF).

The ratio of LF to HF (LF/HF) while the content 120 is provided is referred to as a ratio Rag. A predetermined threshold of the ratio Rag is referred to as a heart rate threshold Pth. As an example, when a proportion of a sum of the amplitude of the high beta wave and the amplitude of the gamma wave of the target person 110 to the total amplitude As while the content 120 is provided is greater than the proportion of the sum of the amplitude of the high beta wave and the amplitude of the gamma wave of the target person 110 to the total amplitude As before the content 120 is provided, and the ratio of LF to HF (LF/HF) while the content 120 is provided is equal to or greater than the heart rate threshold Pth, it can be inferred that a state of irritation, a state of nervousness, or a state of stress of the target person 110 is increasing. When the ratio of LF to HF (LF/HF) is equal to or greater than heart rate threshold Pth, it may be determined that the target person 110 is in a state in which a sympathetic nerve is more dominant than a parasympathetic nerve. When the ratio of LF to HF (LF/HF) is less than the heart rate threshold Pth, it may be determined that the target person 110 is in a state in which the parasympathetic nerve is more dominant than the sympathetic nerve. The heart rate threshold Pth may be 2, may be 3, may be 4, or may be 5.

As an example, when a proportion of a sum of the amplitude of the high beta wave and the amplitude of the gamma wave of the target person 110 to the total amplitude As while the content 120 is provided is greater than the proportion of the sum of the amplitude of the high beta wave and the amplitude of the gamma wave of the target person 110 to the total amplitude As before the content 120 is provided, and the ratio of LF to HF (LF/HF) while the content 120 is provided is less than the heart rate threshold Pth, it can be inferred that a state of excitement of the target person 110 is increasing.

The state estimation unit 20 may estimate the state S based on a magnitude relationship between the ratio Rag and the heart rate threshold Pth, and based on the change C. The proportion of the sum of the amplitude of the high beta wave and the amplitude of the gamma wave of the target person 110 before the content 120 is provided, to the total amplitude As, is referred to as a proportion Ra1. The proportion of the sum of the amplitude of the high beta wave and the amplitude of the gamma wave of the target person 110 while the content 120 is provided, to the total amplitude As, is referred to as a proportion Ra2.

When the proportion Ra2 is greater than the proportion Ra1 and the ratio Rag is equal to or greater than the heart rate threshold Pth, the state estimation unit 20 may estimate that stress of the target person 110 toward the content 120 is increasing. When the proportion Ra2 is greater than the proportion Ra1 and the ratio Rag is less than the heart rate threshold Pth, the state estimation unit 20 may estimate that a degree of immersion of the target person 110 into the content 120 is increasing.

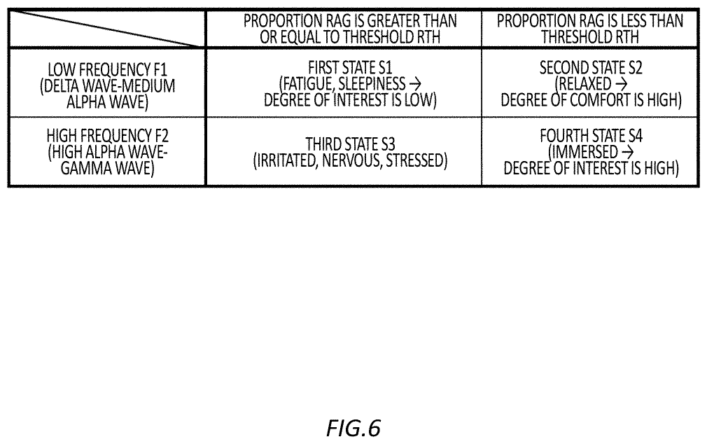

FIG. 6 is a diagram showing an example of the state S of the target person 110, estimated by the state estimation unit 20. The state S may include a plurality of states (a first state S1 to a nth state Sn) of the target person 110. In the present example, the state S includes four states (the first state S1 to a fourth state S4) of the target person 110. In FIG. 6, the brain wave of a low frequency f1 refers to at least one of the delta wave, the theta wave, the low alpha wave, or the medium alpha wave, and the brain wave of a high frequency f2 refers to at least one of the high alpha wave, the low beta wave, the high beta wave, or the gamma wave.

The amplitude of the brain wave of the target person 110, which is the amplitude of the brain wave in the predetermined frequency band, is referred to as an amplitude Af. The amplitude Af of the brain wave of the target person 110 before the content 120 is provided is referred to as an amplitude Af1. The amplitude Af of the brain wave of the target person 110 while the content 120 is provided is referred to as an amplitude Af2. The brain wave in the predetermined frequency band may be at least one of the low alpha wave, the medium alpha wave, the high alpha wave, the low beta wave, the high beta wave, the gamma wave, or the theta wave.

In the present example, the first state S1 is the state S of target person 110 when a proportion of the amplitude Af2 to the total amplitude As is greater than a proportion of the amplitude Af1 to the total amplitude As in the brain wave of the low frequency f1, and the ratio of LF to HF (LF/HF) (the ratio Rag as described above) while the content 120 is provided is equal to or greater than a threshold Rth. When the target person 110 is in the first state S1, it can be inferred that the target person 110 is in the state S in which fatigue or sleepiness of the target person 110 is increasing. Therefore, the state estimation unit 20 may estimate that the state S of the target person 110 is the first state S1 in which a degree of interest in the content 120 is decreasing.

In the present example, a second state S2 is the state S of target person 110 when the proportion of the amplitude Af2 to the total amplitude As is greater than the proportion of the amplitude Af1 to the total amplitude As in the brain wave of the low frequency f1, and the ratio Rag is less than the threshold Rth. When the target person 110 is in the second state S2, it can be inferred that the target person 110 is in the state S in which a degree of relaxation of the target person 110 is increasing. Therefore, the state estimation unit 20 may estimate that the state S of the target person 110 is the second state S2 in which a degree of comfort with the content 120 is increasing.

In the present example, a third state S3 is the state S of target person 110 when the proportion of the amplitude Af2 to the total amplitude As is greater than the proportion of the amplitude Af1 to the total amplitude As in the brain wave of the high frequency f2, and the ratio Rag is equal to or greater than the threshold Rth. When the target person 110 is in the third state S3, it can be inferred that the target person 110 is in the state S in which irritation, nervousness or stress of the target person 110 is increasing. Therefore, the state estimation unit 20 may estimate that the state S of the target person 110 is the third state S3 in which irritation, nervousness or stress toward the content 120 is increasing.

In the present example, the fourth state S4 is the state S of target person 110 when the proportion of the amplitude Af2 to the total amplitude As is greater than the proportion of the amplitude Af1 to the total amplitude As in the brain wave of the high frequency f2, and the ratio Rag is less than the threshold Rth. When the target person 110 is in the fourth state S4, it can be inferred that the target person 110 is in the state S in which the degree of immersion of the target person 110 is increasing. Therefore, the state estimation unit 20 may estimate that the state S of the target person 110 is the fourth state S4 in which the degree of interest in the content 120 is increasing.

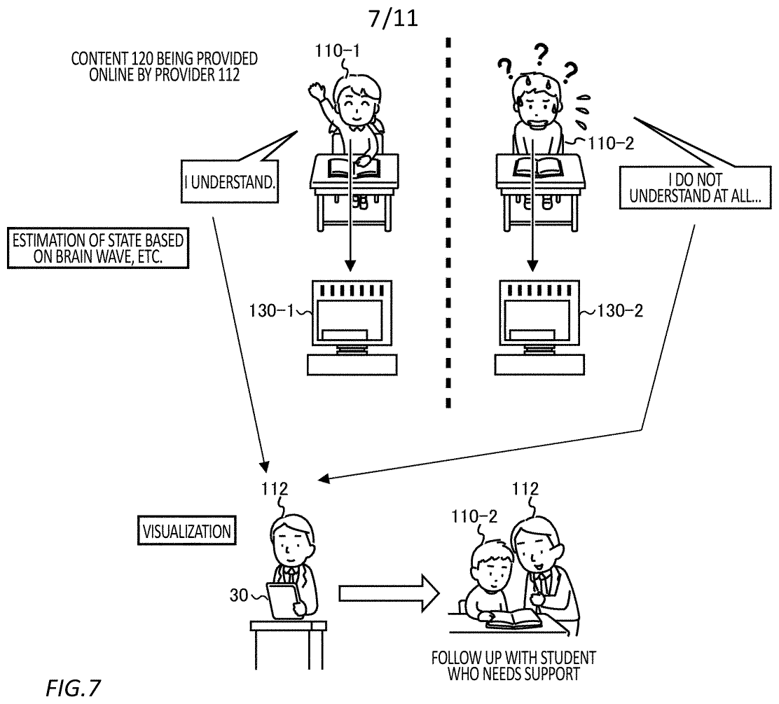

FIG. 7 is a diagram showing another example of the situation in which the content 120 is provided to the target person 110. In the present example, the content 120 is provided online to the plurality of target persons 110. In the example in FIG. 7, the content 120 is provided through a terminal 130-1 of the target person 110-1, and the content 120 is provided through a terminal 130-2 of the target person 110-2. Each of the plurality of target persons 110 may be present in different locations. In the example in FIG. 7, the content 120 is provided to the target person 110-1 and the target person 110-2 at their homes respectively.

The information acquisition unit 10 may acquire the brain wave information Ib of each of the plurality of target persons 110 to whom the content 120 which is common is provided. In the examples in FIG. 1 and FIG. 7, the brain wave information Ib of the target person 110-1 is referred to as brain wave information Ib1, and the brain wave information Ib of the target person 110-2 is referred to as brain wave information Ib2. In the examples in FIG. 1 and FIG. 7, the information acquisition unit 10 acquires the brain wave information Ib1 of the target person 110-1 and the brain wave information Ib2 of the target person 110-2 to whom the content 120 which is common is provided.

The content 120 which is common may be a same lesson, a same lecture, or the like. The content 120 which is common may be video, images or audio relating to a same viewing target.

“The content 120 which is common is provided” may refer to, in a case where the same lesson, the same lecture, or the like is provided to the plurality of target persons 110, a situation in which the same lesson, the same lecture, or the like is provided in a common location (as the example in FIG. 1). “The content 120 which is common is provided” may refer to, in a case where the video, the images, or the audio pertaining to the same lesson, the same lecture, or the like is provided to the plurality of target persons 110, a case where a same video, same images, or a same audio is provided to each of the plurality of target persons 110 present in different locations (as the example in FIG. 7), or may refer to a case where the same video, the same images, or the same audio is provided to the plurality of target persons 110 in the common location.

A case where the same video, the same images, or the same audio is provided to each of the plurality of target persons 110 present in different locations (as the example in FIG. 7) may refer to a case where the same video, the same images, or the same audio is provided to each of the plurality of target persons 110 via the Internet. When the same video, the same images, or the same audio is provided to each of the plurality of target persons 110 via the Internet, the same video, the same images, or the same audio may be provided to each of the plurality of target persons 110 at different times or at the same time. A case where the same video, the same images, or the same audio is provided at different times via the Internet refers to, for example, a case where the content 120 for e-learning is provided to the target person 110.

The state estimation unit 20 may estimate the state S of each of the plurality of target persons 110 based on the brain wave information Ib of each of the plurality of target persons 110. In the examples in FIG. 1 and FIG. 7, the state estimation unit 20 estimates the state S of the target person 110-1 based on the brain wave information Ib1 of the target person 110-1, and estimates the state S of the target person 110-2 based on the brain wave information Ib2 of the target person 110-2.

The content control unit 40 may control the content 120 based on the state S of each of the plurality of target persons 110. The content control unit 40 may change the content 120 according to the state S of each of the plurality of target persons 110. In a case where the video, images or audio pertaining to the same lesson, the same lecture, or the like is provided to the plurality of target persons 110 in different locations (as the example in FIG. 7), the content control unit 40 may change the content 120 to be provided to one target person 110 to the content 120 in accordance with to the state S of the one target person 110, and may change the content 120 to be provided to another target person 110 to the content 120 in accordance with the state S of the another target person 110. The content control unit 40 may cause the content 120 which is to be provided to the one target person 110 to be different from the content 120 which is to be provided to the another target person 110, according to the state S. This allows the content control unit 40 to provide the content 120 in accordance with the state S of each of the plurality of target persons 110. The content 120 controlled by the content control unit 40 may be provided wirelessly via the Internet or the like to each of the target persons 110 present in different locations.

In a case where the state S of the target person 110 is the first state S1 or the third state S3 (see FIG. 6), the content control unit 40 may control the content 120 to provide the target person 110 with the content 120 in accordance with the state S of the target person 110, which is for facilitating understanding. For example, in the case where the content 120 for e-learning is provided to the target person 110, the content control unit 40 may change the content 120 to be provided via the Internet to the content 120 for facilitating understanding, or may add another content 120 for facilitating understanding to the content 120 being provided. Adding the another content 120 for facilitating understanding may include, for example, adding a Uniform Resource Locator (URL) indicating a location of the content 120 for facilitating understanding.

In a case where the state S of the target person 110 is the second state S2 or the fourth state S4 (see FIG. 6), the content control unit 40 may control the content 120 to provide the target person 110 with the content 120 in accordance with the state S of the target person 110, which is more challenging. For example, in the case where the content 120 for e-learning is provided to the target person 110, the content control unit 40 may change the content 120 to be provided via the Internet to the content 120 which is more challenging, or may add the another content 120 which is more challenging to the content 120 being provided.

The content control unit 40 may generate a task for the target person 110 based on the state S of target person 110, and may control the content 120 to provide the generated task to the target person 110. The task based on the state S may refer to a task for facilitating the degree of understanding of the content 120 by the target person 110. The task for the target person 110 may be prepared in advance. A plurality of tasks for the target person 110 may be prepared in advance, each according to a plurality of anticipated states S. The task prepared in advance may be stored in the storage unit 50 (see FIG. 4). The content control unit 40 may select one of the plurality of tasks prepared in advance, according to the state S, and may control the content 120 to provide the selected task to the target person 110.

In the example in FIG. 7, the information acquisition unit 10 may further acquire identification information for identifying the terminal 130 of each of the plurality of target persons 110, to which the content 120 is provided. The identification information is referred to as identification information Id. The content control unit 40 may control the content 120 based on the identification information Id and the state S of each of the plurality of target persons 110. The content control unit 40 may change the content 120 to be provided to each of the plurality of target persons 110 to the content 120 in accordance with each of the states S based on the identification information Id, and may also add the another content 120 for facilitating understanding. Adding the another content 120 for facilitating understanding may include, for example, adding the URL indicating the location of the content 120 for facilitating understanding. This can change the state S of the target person 110 to the second state S2 or the fourth state S4 (see FIG. 6) when the state S of the target person 110 is the first state S1 or the third state S3 (see FIG. 6).

The content control unit 40 may classify the plurality of target persons 110 into stages based on the state S of each of the plurality of target persons 110. Classifying the plurality of target persons 110 into stages refers, for example, to classify the plurality of target persons 110 into groups such as advanced, intermediate, beginner, or the like. The content control unit 40 may control the content 120 to be provided to each group of the plurality of target persons 110 classified into stages, so that the content 120 is in accordance with the state S of the target person 110 at each of the stages. This makes it easier to provide the target persons 110 in each group with the content 120 which is optimal in accordance with the state S.

The providing unit 30 (see FIG. 4) may provide, based on the state S of the target person 110, another content to the provider 112 of the content 120 for bringing the state S of the target person 110 into a predetermined state S. The providing unit 30 may, based on the state S of each of the plurality of target persons 110, provide the another content for bringing the state S of the plurality of target persons 110 into the predetermined state S. The predetermined state S may refer to the second state S2 or the fourth state S4 (see FIG. 6). In a case where the target person 110 is in the first state S1 or in the third state S3 (see FIG. 6), the another content for bringing the target person 110 into the second state S2 or into the fourth state S4 is, for example, “Let's add intonation to your speech,” “Let's explain with a more understandable example”, or the like.

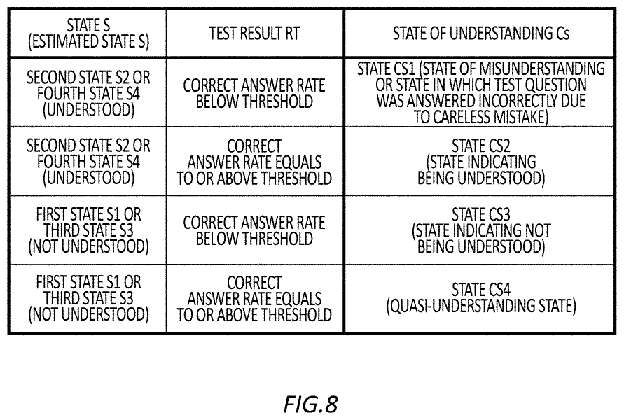

FIG. 8 is a diagram showing an example of a relationship between the state S, a test result Rt and a state of understanding Cs. The state S is the state S of the target person 110 estimated by the state estimation unit 20, as described above. In the present example, the state S is denoted as an estimated state S. The test result Rt is a result of a test on the degree of understanding of the content 120 by the target person 110. The test may be conducted after the content 120 is provided to the target person 110. In the example in FIG. 7, the test may be provided to the terminal 130 of each of the plurality of target persons 110, and each of the plurality of target persons 110 may take the test with the terminal 130. The information acquisition unit 10 may acquire the test result Rt.

The state estimation unit 20 may determine the state of understanding Cs of the content 120 by the target person 110, based on the estimated state S and the test result Rt. The state of understanding Cs may include a plurality of states regarding the understanding of the content 120 by the target person 110. In the present example, the state of understanding Cs includes four states from a state Cs1 to a state Cs4.

When the estimated state S is the second state S2 or the fourth state S4 (a state indicating being understood) and the test result Rt is less than a predetermined correct answer rate threshold, the state estimation unit 20 may determine that the state of understanding Cs by the target person 110 is a state in which the target person 110 misunderstands the content 120 or a state in which the target person 110 answered a test question incorrectly due to a careless mistake. This state is the state Cs1. The predetermined correct answer rate threshold may be, for example, 70% correct, may be 80% correct, or may be 90% correct. When the estimated state S is the second state S2 or the fourth state S4 (a state indicating being understood) and the test result Rt is equal to or greater than the predetermined correct answer rate threshold, the state estimation unit 20 may determine that the state of understanding Cs by the target person 110 is a state in which the target person 110 understands the content 120. This state is the state Cs2.

When the estimated state S is the first state S1 or the third state S3 (a state indicating not being understood) and the test result Rt is less than the predetermined correct answer rate threshold, the state estimation unit 20 may determine that the state of understanding Cs by the target person 110 is a state in which the target person 110 does not understand the content 120. This state is the state Cs3.

When the estimated state S is the first state S1 or the third state S3 (a state indicating not being understood) and the test result Rt is equal to or greater than the predetermined correct answer rate threshold, the state estimation unit 20 may determine that the state of understanding Cs by the target person 110 is a quasi-understanding state. The quasi-understanding state is referred to as a quasi-understanding state Cs4. The quasi-understanding state Cs4 is described below.

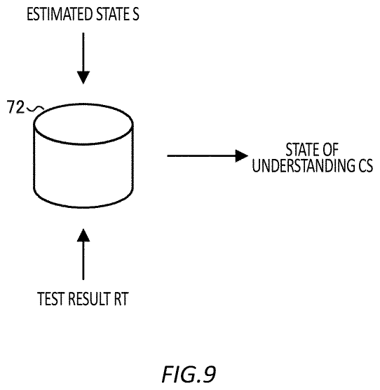

FIG. 9 is a diagram showing an example of a state of understanding inference model 72. The state learning unit 70 (see FIG. 4) performs machine learning on a relationship between the estimated state S of each of the plurality of target persons 110 and the test result Rt of each of the plurality of target persons 110. The state learning unit 70 generates the state of understanding inference model 72 by performing machine learning on the relationship between the estimated state S of each of the plurality of target persons 110 and the test result Rt of each of the plurality of target persons 110. The state of understanding inference model 72 infers the state of understanding Cs based on the estimated state S. The state of understanding inference model 72 may be stored in the storage unit 50.

The state learning unit 70 performs machine learning on the relationship between the estimated state S and the test result Rt. Therefore, the state of understanding Cs inferred by the state of understanding inference model 72 is highly likely to be a true state of understanding Cs by the target person 110. The state of understanding Cs inferred by the state of understanding inference model 72 may be provided by the providing unit 30. This makes it easier for the provider 112 of the content 120 to recognize the true state of understanding Cs by the target person 110.

When the state learning unit 70 generates the state of understanding inference model 72 by performing machine learning on the estimated state S pertaining to the quasi-understanding state Cs4 and the test result Rt, the state estimation unit 20 may estimate that the quasi-understanding state Cs4 is the state in which the target person 110 understands the content 120. A case where the state learning unit 70 generates the state of understanding inference model 72 by performing machine learning on the estimated state S pertaining to the quasi-understanding state Cs4 and the test result Rt refers to a case where the quasi-understanding state Cs4 can be estimated to be the state in which the target person 110 understands the content 120, because there are a certain number of cases determined to be the quasi-understanding state Cs4. In a case where the quasi-understanding state Cs4 is estimated to be the state in which the target person 110 understands the content 120, when the state estimation unit 20 estimates the estimated state S pertaining to the quasi-understanding state Cs4 based on the brain wave information Ib, the brain wave information Ib may be estimated to be the brain wave information Ib when the target person 110 understands the content 120.

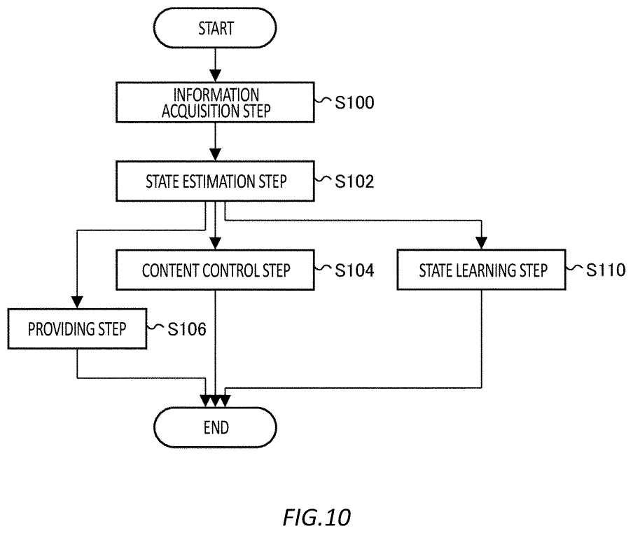

FIG. 10 is a flowchart showing an example of a state estimation method according to an embodiment of the present invention. The state estimation method according to the embodiment of the present invention is described using a state estimation apparatus 100 shown in FIG. 4 as an example. The state estimation method includes an information acquisition step S100 and a state estimation step S102. The state estimation method may include a content control step S104, a providing step S106, an evaluation step S108, and a state learning step S110.

The information acquisition step S100 is a step in which the information acquisition unit 10 acquires the brain wave information Ib of the target person 110 to whom the content 120 is provided. The state estimation step S102 is a step in which the state estimation unit 20 estimates the state S of the target person 110 to whom the content 120 is provided, based on the brain wave information Ib of the target person 110.

The information acquisition step S100 may be a step in which the information acquisition unit 10 further acquires the biometric information Ig of the target person 110 to whom the content 120 is provided. The state estimation step S102 may be a step in which the state estimation unit 20 estimates the state S based on the brain wave information Ib and the biometric information Ig.

The information acquisition step S100 may be a step in which the information acquisition unit 10 acquires the brain wave information Ib of the target person 110 before and while the content 120 is provided. The state estimation step S102 may be a step in which the state estimation unit 20 estimates a state based on a change from the brain wave information Ib before the content 120 is provided to the brain wave information Ib while the content 120 is provided, and based on the biometric information Ig.

The state estimation step S102 may be a step in which the state estimation unit 20 estimates the state S based on the change (the change C as described above) from a proportion of an amplitude Af1 to the total amplitude As in the brain wave information Ib before the content 120 is provided, to a proportion of an amplitude Af2 to the total amplitude As in the brain wave information Ib while the content 120 is provided, and based on the ratio of LF to HF (LF/HF) while the content 120 is provided (the ratio Rag as described above). The state estimation step S102 may be a step in which the state estimation unit 20 estimates the state S based on the magnitude relationship between the ratio Rag and the heart rate threshold Pth, and based on the change C.

The information acquisition step S100 may be a step in which the information acquisition unit 10 acquires the brain wave information Ib of each of the plurality of target persons 110 to whom the content 120 which is common is provided. The state estimation step S102 may be a step in which the state estimation unit 20 estimates the state S of each of the plurality of target persons 110, based on the brain wave information Ib of each of the plurality of target persons 110. The content control step S104 is a step in which the content control unit 40 controls the content 120 based on the state S of each of the plurality of target persons 110.

The information acquisition step S100 may be a step in which the information acquisition unit 10 further acquires the identification information Id for identifying the terminal 130 of each of the plurality of target persons 110, to which the content 120 is provided. The content control step S104 may be a step in which the content control unit 40 controls the content 120 based on the identification information Id and the state S of each of the plurality of target persons 110. The content control step S104 may be a step in which the content control unit 40 classifies the plurality of target persons 110 into stages based on the state S of each of the plurality of target persons 110, and controls the content 120 to be provided to each group of the plurality of target persons 110 classified into stages, so that the content 120 is in accordance with the state S at each of the stages.

The providing step S106 is a step in which the providing unit 30 provides, based on the state S of the target person 110, the another content to the provider 112 of the content 120 for bringing the state S of the target person 110 into the predetermined state S.

The information acquisition step S100 may be a step in which the information acquisition unit 10 further acquires the test result Rt regarding the degree of understanding of the content 120 by the target person 110. The state estimation step S102 may be a step in which the state of understanding Cs of the content 120 by the target person 110 is determined based on the estimated state which is the state S estimated by the state estimation unit 20, and based on the test result Rt.

The state learning step S110 is a step in which the state learning unit 70 generates the state of understanding inference model 72, which infers the state of understanding Cs based on the estimated state S, by performing machine learning on the relationship between the estimated state S of each of the plurality of target persons 110 and the test result Rt of each of the plurality of target persons 110.

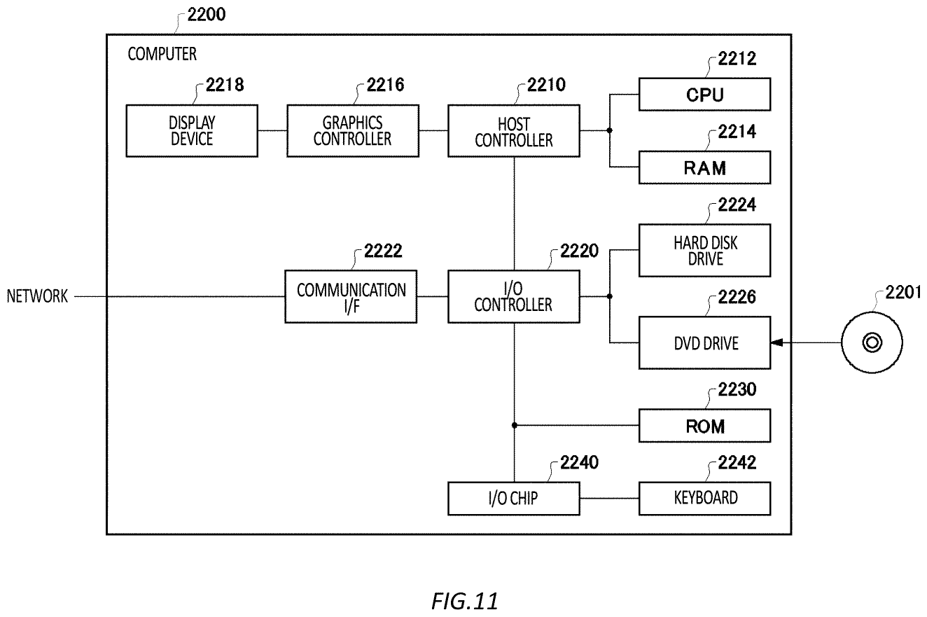

FIG. 11 is a diagram showing an example of a computer 2200, in which the state estimation apparatus 100 according to an embodiment of the present invention may be embodied in whole or in part. A program installed in the computer 2200 can cause the computer 2200 to perform operations associated with the state estimation apparatus 100 according to the embodiment of the present invention, or to function as one or more sections of the state estimation apparatus 100, or can cause the computer 2200 to perform the operations or the one or more sections, or can cause the computer 2200 to perform each step (see FIG. 10) according to the state estimation method of the present invention. The program may be executed by a CPU 2212 to cause the computer 2200 to perform certain operations associated with some or all of the blocks of flowcharts (see FIG. 10) and block diagrams (see FIG. 4) described herein.

The computer 2200 according to an embodiment of the present invention includes the CPU 2212, a RAM 2214, a graphics controller 2216, and a display device 2218. The CPU 2212, the RAM 2214, the graphics controller 2216, and the display device 2218 are mutually connected by a host controller 2210. The computer 2200 further includes input/output unit such as a communication interface 2222, a hard disk drive 2224, a DVD-ROM drive 2226, and an IC card drive. The communication interface 2222, the hard disk drive 2224, the DVD-ROM drive 2226, and the IC card drive, and the like are connected to the host controller 2210 via an input/output controller 2220. The computer further includes legacy input/output unit such as a ROM 2230 and a keyboard 2242. The ROM 2230, the keyboard 2242, and the like are connected to the input/output controller 2220 via an input/output chip 2240.

The CPU 2212 operates according to a program stored in the ROM 2230 and the RAM 2214, thereby controlling each unit. The graphics controller 2216 acquires image data generated by the CPU 2212 on a frame buffer or the like provided in the RAM 2214 or in the RAM 2214 itself to cause the image data to be displayed on the display device 2218.

The communication interface 2222 communicates with other electronic devices via a network. The hard disk drive 2224 stores programs and data used by the CPU 2212 in the computer 2200. The DVD-ROM drive 2226 reads the programs or the data from the DVD-ROM 2201, and provides the read programs or data to the hard disk drive 2224 via the RAM 2214. The IC card drive reads programs and data from an IC card, or writes programs and data to the IC card.

The ROM 2230 stores a boot program or the like executed by the computer 2200 at the time of activation, or a program depending on the hardware of the computer 2200. The input/output chip 2240 may connect various input/output unit via a parallel port, a serial port, a keyboard port, a mouse port, or the like to the input/output controller 2220.

Programs are provided by a computer readable medium such as the DVD-ROM 2201 or the IC card. The programs are read from the computer readable medium, are installed in the hard disk drive 2224, the RAM 2214, or the ROM 2230 which is also an example of the computer readable medium, and are executed by the CPU 2212. The information processing described in these programs is read by the computer 2200, and provides cooperation between the programs and the various types of above-described hardware resources. An apparatus or a method may be constituted by realizing the operation or processing of information in accordance with the usage of the computer 2200.

For example, when a communication is executed between the computer 2200 and an external device, the CPU 2212 may execute a communication program loaded onto the RAM 2214 to instruct communication processing to the communication interface 2222, based on the processing written in the communication program. The communication interface 2222, under control of the CPU 2212, reads transmission data stored on a transmission buffering region provided in a recording medium such as the RAM 2214, the hard disk drive 2224, the DVD-ROM 2201, or the IC card, and transmits the read transmission data to a network or writes reception data received from a network to a reception buffering region or the like provided on the recording medium.

The CPU 2212 may cause all or a necessary portion of a file or a database to be read into the RAM 2214, the file or the database having been stored in an external recording medium such as the hard disk drive 2224, the DVD-ROM drive 2226 (DVD-ROM 2201), the IC card, or the like. The CPU 2212 may execute various types of processing on the data on the RAM 2214. The CPU 2212 may then write back the processed data to the external recording medium.

Various types of information, such as various types of programs, data, tables, and databases, may be stored in the recording medium to undergo information processing. The CPU 2212 may execute various types of processing on the data read from the RAM 2214, which includes various types of operations, information processing, conditional judgement, conditional branching, unconditional branching, search or replace of information, or the like, as described throughout the present disclosure and designated by an instruction sequence of programs. The CPU 2212 may write the result back to the RAM 2214.

The CPU 2212 may search for information in a file, a database, or the like in the recording medium. For example, when a plurality of entries, each having an attribute value of a first attribute associated with an attribute value of a second attribute, are stored in the recording medium, the CPU 2212 may search for an entry matching the condition, attribute value of the first attribute of which is designated, from among the plurality of entries, read the attribute value of the second attribute stored in the entry, and read a second attribute value to acquire the attribute value of the second attribute associated with the first attribute satisfying the predetermined condition.

The program or software modules described above may be stored in the computer readable medium on the computer 2200 or of the computer 2200. A recording medium such as a hard disk or a RAM provided in a server system connected to a dedicated communication network or the Internet can be used as the computer readable media. The program may be provided to the computer 2200 by the recording medium.

While the present invention has been described above by way of the embodiments, the technical scope of the present invention is not limited to the scope described in the above-described embodiments. It is apparent to persons skilled in the art that various alterations or improvements can be made to the above-described embodiments. It is also apparent from the described scope of the claims that the embodiments added with such alterations or improvements can also be included the technical scope of the present invention.

It should be noted that, the operations, procedures, steps, stages, and the like of each process performed by an apparatus, system, program, and method shown in the claims, specifications, and diagrams can be realized in any order as long as the order is not indicated by “prior to,” “before,” or the like and as long as the output from a previous process is not used in a later process. Even if the operational flow is described using phrases such as “first” or “next” for convenience in the claims, specifications, and diagrams, it does not necessarily mean that the process must be performed in this order.

### EXPLANATION OF REFERENCES

10: information acquisition unit,
20: state estimation unit,
30: providing unit,
40: content control unit,
50: storage unit,
70: state learning unit,
72: state of understanding inference model,
90: control unit,
100: state estimation apparatus,
110: target person,
112: provider,
120: content,
130: terminal,
2200: computer,
2201: DVD-ROM,
2210: host controller,
2212: CPU,
2214: RAM,
2216: graphics controller,
2218: display device,
2220: input/output controller,
2222: communication interface,
2224: hard disk drive,
2226: DVD-ROM drive,
2230: ROM,
2240: input/output chip,
2242: keyboard.

What is claimed is:

1. A state estimation apparatus comprising:
an information acquisition unit which acquires brain wave information of a target person to whom a content is provided; and
a state estimation unit which estimates a state of the target person to whom the content is provided, based on the brain wave information of the target person.

2. The state estimation apparatus according to claim 1, wherein:
the information acquisition unit further acquires biometric information of the target person to whom the content is provided; and
the state estimation unit estimates the state based on the brain wave information and the biometric information.

3. The state estimation apparatus according to claim 2, wherein:
the information acquisition unit acquires the brain wave information of the target person before and while the content is provided; and
the state estimation unit estimates the state based on a change from the brain wave information before the content is provided, to the brain wave information while the content is provided, and based on the biometric information.

4. The state estimation apparatus according to claim 3, wherein:
the state estimation unit estimates the state based on a change from a proportion of an amplitude of a brain wave in a predetermined frequency band to a total amplitude in the brain wave information before the content is provided, to a proportion of an amplitude of a brain wave in the predetermined frequency band to the total amplitude in the brain wave information while the content is provided, and based on a ratio of a magnitude of a first power spectrum to a magnitude of a second power spectrum in a heart rate of the target person; wherein
the total amplitude is a sum of an amplitude of an alpha wave, an amplitude of a beta wave, an amplitude of a theta wave, an amplitude of a gamma wave, and an amplitude of a delta wave; and
a frequency band of the second power spectrum is a band in which a frequency is higher than that in a frequency band of the first power spectrum.

5. The state estimation apparatus according to claim 4, wherein:
the state estimation unit estimates the state based on a magnitude relationship between a ratio of a magnitude of the first power spectrum to a magnitude of the second power spectrum while the content is provided and a predetermined heart rate threshold of a ratio of a magnitude of the first power spectrum to a magnitude of the second power spectrum, and based on the change from the proportion of the amplitude of the brain wave in the predetermined frequency band to the total amplitude before the content is provided, to the proportion of the amplitude of the brain wave in the predetermined frequency band to the total amplitude while the content is provided.

6. The state estimation apparatus according to claim 1, wherein:
the information acquisition unit acquires the brain wave information of each of a plurality of target persons, the target person includes a plurality of target persons, to whom the content which is common is provided; and
the state estimation unit estimates the state of each of the plurality of target persons based on the brain wave information of each of the plurality of target persons; and the state estimation apparatus further comprises:
a content control unit which controls the content based on the state of each of the plurality of target persons.

7. The state estimation apparatus according to claim 2, wherein:
the information acquisition unit acquires the brain wave information of each of a plurality of target persons, the target person includes a plurality of target persons, to whom the content which is common is provided; and
the state estimation unit estimates the state of each of the plurality of target persons based on the brain wave information of each of the plurality of target persons; and the state estimation apparatus further comprises:
a content control unit which controls the content based on the state of each of the plurality of target persons.

8. The state estimation apparatus according to claim 3, wherein:
the information acquisition unit acquires the brain wave information of each of a plurality of target persons, the target person includes a plurality of target persons, to whom the content which is common is provided; and
the state estimation unit estimates the state of each of the plurality of target persons based on the brain wave information of each of the plurality of target persons; and the state estimation apparatus further comprises:
a content control unit which controls the content based on the state of each of the plurality of target persons.

9. The state estimation apparatus according to claim 4, wherein:
the information acquisition unit acquires the brain wave information of each of a plurality of target persons, the target person includes a plurality of target persons, to whom the content which is common is provided; and
the state estimation unit estimates the state of each of the plurality of target persons based on the brain wave information of each of the plurality of target persons; and the state estimation apparatus further comprises:
a content control unit which controls the content based on the state of each of the plurality of target persons.

10. The state estimation apparatus according to claim 5, wherein:
the information acquisition unit acquires the brain wave information of each of a plurality of target persons, the target person includes a plurality of target persons, to whom the content which is common is provided; and
the state estimation unit estimates the state of each of the plurality of target persons based on the brain wave information of each of the plurality of target persons; and the state estimation apparatus further comprises:
a content control unit which controls the content based on the state of each of the plurality of target persons.

11. The state estimation apparatus according to claim 6, wherein
the information acquisition unit further acquires identification information for identifying a terminal of each of the plurality of target persons, to which the content is provided; and
the content control unit controls the content based on the identification information and the state of each of the plurality of target persons.

12. The state estimation apparatus according to claim 11, wherein
the content control unit classifies the plurality of target persons into stages based on the state of each of the plurality of target persons, and controls the content to be provided to each group of the plurality of target persons classified into the stages, so that the content is in accordance with the state at each of the stages.

13. The state estimation apparatus according to claim 6, further comprising:
a providing unit which, based on the state of the target person, provides another content to a provider of the content to bring the state of the target person into a predetermined state.

14. The state estimation apparatus according to claim 1, wherein:
the information acquisition unit further acquires a test result regarding a degree of understanding of the content by the target person; and
the state estimation unit determines a state of understanding of the content by the target person based on an estimated state which is the state estimated by the state estimation unit, and based on the test result.

15. The state estimation apparatus according to claim 2, wherein:
the information acquisition unit further acquires a test result regarding a degree of understanding of the content by the target person; and
the state estimation unit determines a state of understanding of the content by the target person based on an estimated state which is the state estimated by the state estimation unit, and based on the test result.

16. The state estimation apparatus according to claim 3, wherein:
the information acquisition unit further acquires a test result regarding a degree of understanding of the content by the target person; and
the state estimation unit determines a state of understanding of the content by the target person based on an estimated state which is the state estimated by the state estimation unit, and based on the test result.

17. The state estimation apparatus according to claim 4, wherein:
the information acquisition unit further acquires a test result regarding a degree of understanding of the content by the target person; and
the state estimation unit determines a state of understanding of the content by the target person based on an estimated state which is the state estimated by the state estimation unit, and based on the test result.

18. The state estimation apparatus according to claim 14, further comprising:
a state learning unit which generates a state of understanding inference model, which infers the state of understanding based on the estimated state, by performing machine learning on a relationship between the estimated state of each of a plurality of target persons, the target person includes a plurality of target persons, and the test result of each of the plurality of target persons.

19. A state estimation method comprising:
acquiring, by an information acquisition unit, brain wave information of a target person to whom a content is provided; and
estimating, by a state estimation unit, a state of the target person to whom the content is provided, based on the brain wave information of the target person.

20. A non-transitory computer-readable medium having recorded thereon a state estimation program that, when executed by a computer, causes the computer to perform operations comprising:
acquiring brain wave information of a target person to whom a content is provided; and
estimating a state of the target person to whom the content is provided, based on the brain wave information of the target person.
